(19) United States
(12) Patent Application Publication
(10) Pub. No.: US 2025/0340817 A1
(43) Pub. Date: Nov. 6, 2025

(54) PASSIVE CONDENSATION MANAGEMENT FOR AN EXHAUST GAS STREAM IN A BIOPROCESSING SYSTEM

(71) Applicant: Global Life Sciences Solutions USA LLC, Marlborough, MA (US)

(72) Inventors: Colin Tuohey, Marlborough, MA (US); Jeffrey Scott Socha, Marlborough, MA (US); Nicholas Brocato, Marlborough, MA (US); Richard Damren, Marlborough, MA (US)

(73) Assignee: Global Life Sciences Solutions USA LLC, Marlborough, MA (US)

(21) Appl. No.: 18/654,093

(22) Filed: May 3, 2024

(51) Int. Cl. C12M 1/00; C12M 1/12; C12M 1/34

(52) U.S. Cl. CPC C12M 29/20; C12M 25/02; C12M 41/12

(57) ABSTRACT

A method for removing moisture from a gas stream from a bioprocessing vessel includes the steps of passing an exhaust gas through a condensing chamber, the condensing chamber being passively cooled by an ambient environment within which the condensing chamber is located, a temperature of the condensing chamber causing moisture within the exhaust gas to condense within the condensing chamber to produce condensate, and passing the exhaust gas through at least one filter device.

### BACKGROUND

### Technical field

Embodiments of the invention relate generally to bioprocessing systems and methods and, more particularly, to methods and systems for passive condensation management in the exhaust gas stream of a bioprocessing system.

### Discussion of Art

Cell culturing is an essential step in manufacturing biological products, and may be carried out in disposable, single-use bioreactors or in non-disposable bioreactors such as steel tank vessels. Perhaps most commonly, a flexible, single-use, bioprocessing container or bag is supported by an outer rigid structure such as a stainless steel shell or vessel. The bag may be positioned within the rigid vessel and filled with the desired fluid for culturing. An agitator assembly disposed within the bag is used to mix the fluid. Regardless of configuration, oxygen to promote cell growth is continuously or intermittently supplied to the fluid via one or more sparging devices, and carbon dioxide is removed from the headspace within the bioreactor above the processing volume. Due to the humid environment within the bioreactor, a gas stream leaving the bioreactor (e.g., the gas removed from the headspace) may contain moisture entrained within the gas stream. The moisture in the gas stream may condense as the gas stream passes through a filter or other system components. This moisture and/or condensation may be detrimental to the functioning of the filter or other system components, and can create undesirable back-pressure on the single-use bioprocessing bags. Moreover, in certain situations foam and/or aerosolized cell debris and/or aerosolized protein matter from the bioreactor can be carried into the exhaust stream, further contributing to filter fouling.

Previous attempts to address the issue of condensation and filter fouling have involved utilizing redundant filters along the exhaust gas line, or active cooling of the exhaust gas stream before it reaches the filters, to remove moisture (e.g., using plant chilled water or a local cooling TCU). These approaches, however, can be costly. In particular, the use of redundant filters, each of which are quite expensive, increases the cost of the bioprocessing system, and operation thereof, as a whole. In addition, active cooling approaches require water-cooled temperature control units to cool the exhaust gas stream, which are heavy, have quite a large footprint, and are costly to purchase and operate.

In view of the above, there is a need for a system and method for managing the condensation in the exhaust gas stream of bioprocessing system and, namely, a system and method that reduces the moisture content of a moisture-containing gas stream within a bioreactor system before it passes to a filter or other system components.

### BRIEF DESCRIPTION

In an embodiment of the invention, a method for removing moisture from a gas stream from a bioprocessing vessel is provided. The method includes the steps of passing an exhaust gas through a condensing chamber, the condensing chamber being passively cooled by an ambient environment within which the condensing chamber is located, a temperature of the condensing chamber causing moisture within the exhaust gas to condense within the condensing chamber to produce condensate, and passing the exhaust gas through at least one filter device.

In another embodiment of the invention, a system for condensation management in a bioprocessing system is provided. The system includes a condensing chamber having an inlet configured for fluid connection with a headspace of a bioprocessing vessel, and an outlet configured for fluid connection with an exhaust filter. The condensing chamber is configured to be passively cooled by an ambient environment within which the condensing chamber and the bioprocessing vessel are located.

In yet another embodiment of the invention, a method for removing moisture from a gas stream during a bioprocessing operation is provided. The method includes the steps of removing an exhaust gas from a headspace within a bioprocessing vessel, and passing the exhaust gas through a condensing chamber, the condensing chamber being passively cooled by an ambient environment within which the condensing chamber is located, a temperature of the condensing chamber causing moisture within the exhaust gas to condense within the condensing chamber to produce condensate. The method further includes passing the exhaust gas through at least one filter device, and routing the collected condensate from the condensing chamber into the bioprocessing vessel.

### BRIEF DESCRIPTION OF DRAWINGS

The present invention will be better understood from reading the following description of non-limiting embodiments, with reference to the attached drawings, wherein below:

FIG. 1 is a schematic illustration of a bioprocessing system having a system for condensation management, according to an embodiment of the invention.

FIG. 2 is a perspective view of a condensing chamber housing for use with the bioprocessing system of FIG. 1, according to an embodiment of the invention.

FIG. 3 is a perspective view of a condensing chamber housing for use with the bioprocessing system of FIG. 1, according to another embodiment of the invention.

FIG. 4 is a simplified schematic illustration of an exhaust gas flowpath through the condensing chamber housing of FIG. 2.

FIG. 5 is a perspective view of a condensing chamber housing for use with the bioprocessing system of FIG. 1, according to another embodiment of the invention.

FIG. 6 is an enlarged, side elevational view of a portion of the condensing chamber housing of FIG. 5.

FIG. 7 is an enlarged, front view of a portion of the condensing chamber housing of FIG. 5.

FIG. 8 is a perspective view of a condensing chamber housing for use with the bioprocessing system of FIG. 1, according to another embodiment of the invention.

FIG. 9 is a simplified, perspective view of a condensing chamber for use with the bioprocessing system of FIG. 1, according to another embodiment of the invention.

FIG. 10 is a simplified, schematic illustration of a portion of a condensing system for use with the bioprocessing system of FIG. 1, according to another embodiment of the invention.

FIG. 11 is a simplified, schematic illustration of a portion of a condensing system for use with the bioprocessing system of FIG. 1, according to another embodiment of the invention.

FIG. 12 is a perspective view of a condensing chamber housing incorporating fans for convection airflow, according to an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “bioreactor” as used herein generally refers to a device or apparatus in the form of a closed chamber or vessel in which living organisms such as mammalian cells, bacteria or yeast synthesize substances useful, for example, to the pharmaceutical industry under controlled conditions favorable to that specific organism. Traditionally bioreactors were closed, rigid stainless-steel vessels in which the organisms were grown. The term “bioreactor” as used herein may be rigid or disposable, single-use bioreactor.

The term “disposable” or “single-use” as used herein in the context of a bioreactor generally refers to a flexible container, liner or bag incorporating all of the functional aspects required of a traditional bioreactor which can be filled with the materials required for the growth of mammalian cells, bacteria or yeast and is designed with the intention that it be disposed of at the completion of a single production run.

The term “moisture-containing gas stream” as used herein generally refers to a gas stream entering or leaving a bioreactor and contains moisture entrained within the moisture-containing gas stream. The moisture-containing gas stream may be referred to as a “gas stream” or a “moist gas stream”. As used herein, “dry gas” of “dry gas stream” means a gas having a reduced moisture content as compared to a moisture content of the gas prior to processing or treatment using the systems and method described herein, and does not necessarily mean a gas that is entirely devoid of moisture (although a gas devoid of moisture or having a nominal moisture content is also contemplated within the definition of “dry gas”).

As used herein, “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

Embodiments of the invention provide systems and method for passive condensation management for exhaust gas streams of a bioprocessing system. Embodiments of the invention include passing an exhaust gas through a condensing chamber, the condensing chamber being passively cooled by an ambient environment within which the condensing chamber is located, a temperature of the condensing chamber causing moisture within the exhaust gas to condense within the condensing chamber to produce condensate, and passing the exhaust gas through at least one filter device.

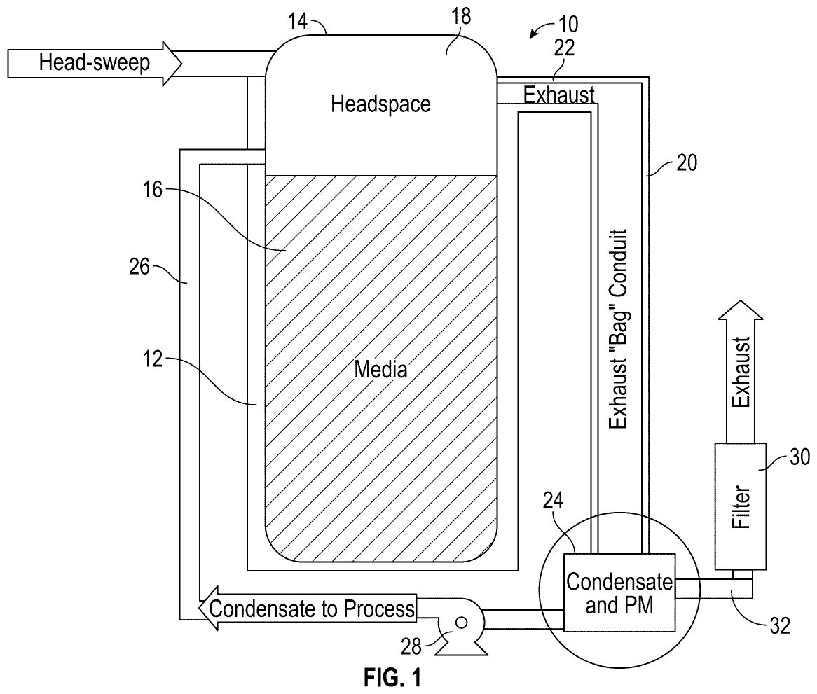

With reference to FIG. 1, a bioprocessing system 10 according to an embodiment of the invention is illustrated. The bioprocessing system 10 includes a generally rigid bioreactor vessel or support structure 12. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly (not shown) that provides support to a single-use, flexible bag 14 disposed within the vessel 12. The vessel 12 can be any shape or size as long as it is capable of supporting a single-use flexible bioreactor bag 14. For example, according to one embodiment of the invention the vessel 12 is capable of accepting and supporting a 10-2000 L flexible or collapsible bioprocess bag assembly 14.

While not shown, the vessel 12 may include one or more sight windows, which allows one to view a fluid level within the flexible bag, as well as a window positioned at a lower area of the vessel 12 which allows access to the interior of the vessel 12 for insertion and positioning of various sensors and probes (not shown) within the flexible bag 14, and for connecting one or more fluid lines to the flexible bag 14 for fluids, gases, and the like, to be added or withdrawn from the flexible bag. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

In an embodiment, the single-use, flexible bag 14 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low-density polyethylene and ultra-low-density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem™, Bioclear™ 10 and Bioclear 11 laminates, available from Cytiva. In an embodiment the flexible material may be ReadyKleer® film, availanle from Cytiva. Portions of the flexible container can comprise a substantially rigid material such as a rigid polymer, for example, high density polyethylene, metal, or glass. The flexible bag may be supplied pre-sterilized, such as using gamma irradiation.

As indicated above, the flexible bag 14 defines therein a processing volume containing media 16 for bioprocessing operations, and a headspace 18 above the processing volume. While not shown, in an embodiment, the flexible bag 14 contains an impeller (e.g., a magnetically driven impeller) for mixing or agitating the media 16, and one or more sparge devices for introducing gas (e.g., oxygen) into the media 16 to support bioprocessing operations.

As best shown in FIG. 1, the bioprocessing system 10 includes a condensing chamber 20 that is in fluid communication with the headspace 18 of the bioreactor (in this case, the flexible bag 14) via an exhaust line 22. A condensate accumulator 24 is located at the bottom of the condensing chamber 20 and is fluidly connected thereto for receiving condensate and/or particulate matter from the condensing chamber 20, as described in detail below. As further shown in FIG. 1, a return line 26 extends from the condensate accumulator 24 back to the bioreactor (i.e., back to the headspace 18 of the flexible bag 14), thus placing the condensate accumulator 24 in fluid communication with the flexible bag 14. In an embodiment, a pump 28 is positioned along the return line 26 to pump condensate back into the processing volume, as described hereinafter. Additionally, one or more filters 30 are fluidly connected to the condensate chamber 24 via a fluid line 32 and receives a dry gas from the condensate chamber 24, which is then exhausted to atmosphere or further treated and/or recycled, as desired and depending on application. In an embodiment, the filters 30 may be any type of filter known in the art. One or more valves (not shown) may be present in one or more of the fluid lines to control the flow of fluid and/or gas therethrough, as desired.

In operation, a relatively hot moisture-containing gas stream leaving the bioreactor 10 exits from the headspace 18 of the bioreactor and enters the condensing chamber 20 via the exhaust line 22. The moisture in the gas stream is condensed inside the condensing chamber 20 to form a relatively cool condensate, which is collected in accumulator 24. A dry gas stream (from which moisture has been removed to form the condensate) flows out of the condensing chamber 20 and condensate accumulator 24 and enters the filter 30 via fluid line 32.

In an embodiment, after the relatively cool condensate is generated in the contact condensing chamber 20 and drains into the condensate accumulator 24 under the force of gravity, it flows to the pump 28. The pump 28 then pumps the condensate back into the bioreactor 10 via the return line 26. In another embodiment, rather than being pumped back to the bioreactor, the condensate can be drained and disposed of.

In an embodiment, the condensing chamber 20 is configured as a two- dimensional, thin-walled, flexible bag or conduit having a front panel and a back panel joined about their respective lateral edges. In a further embodiment, the condensing chamber 20 may be a three-dimensional, thin-walled flexible bag. In an embodiment, the condensing chamber 20 is formed from the same or similar material as the flexible bag 14. In an embodiment, the condensing chamber 20 is placed within the same ambient environment as the bioreactor 10 (e.g., in an ambient environment between about 20-22° C.), which maintains the panels of the condensing chamber 20 at about the same temperature. In this respect, the condensing chamber 20 and the walls thereof are passively cooled. As used herein, “passively cooled” means cooled via contact with the ambient environment within which the bioreactor 10 is located (i.e., without active cooling or chilling devices, such as temperature control units (TCUs) and the like).

In operation, as the warm exhaust gas (exiting the flexible bag 14 at between about 35-40° C. and, more particularly, about 37° C.) enters the condensing chamber 20, it contacts the comparatively cool walls thereof. The larger surface area within the condensing chamber 20, as compared to the exhaust line 22, and the cool walls within the condensing chamber 20, causes the moisture within the exhaust gas stream to condense and form condensate, which is collected in the condensate accumulator 24, as discussed above. The now dry gas stream can be exhausted to the ambient environment after passing through the filter(s) 30 as also disclosed above.

In an embodiment, the condensing chamber 20 is formed or constructed so as to define a tortuous and/or serpentine-like pathway through the condensing chamber 20, which increases contact of the moist exhaust gas stream with the walls of the condensing chamber 20, thereby facilitating condensation and moisture removal. For example, the condensing chamber 20 may include a series of welds that define the tortuous pathway within the flexible condensing chamber 20. Once moisture condenses, it more efficiently collects moisture from gas that is flowing through the tortuous path. Further, foam and/or aerosols (e.g., aerosolized cell debris and/or aerosolized protein matter from the bioreactor) can be collected by impaction (the larger the particle size, the less likely it can navigate the tortuous pathway and will impact on the impaction features of the flexible bag 20). The aerosol laden condensate travels to a low point within the exhaust management system as discussed above, and can then be pumped back into the process bag or be disposed of efficiently, as desired. In this manner, the condensate management system of the invention provides for passive moisture removal, that is, without active cooling of the condensing chamber.

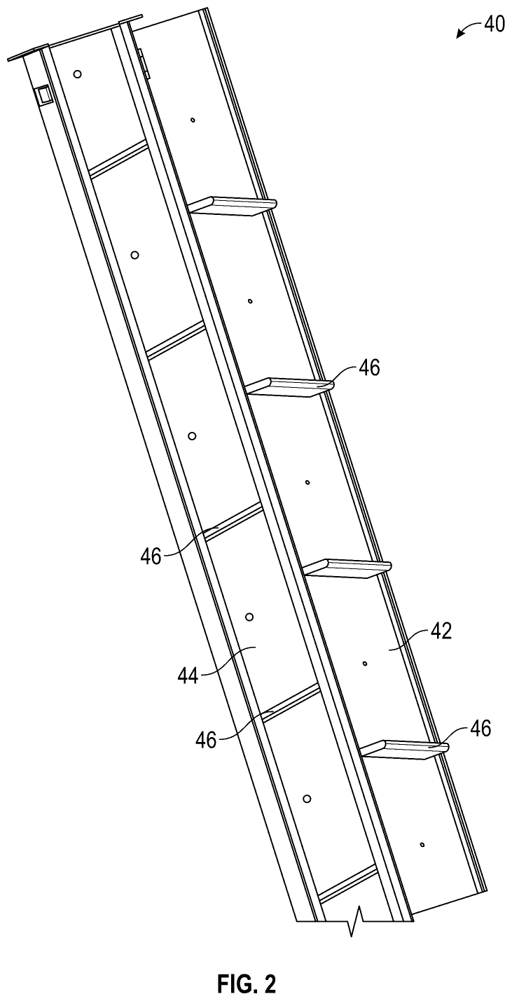

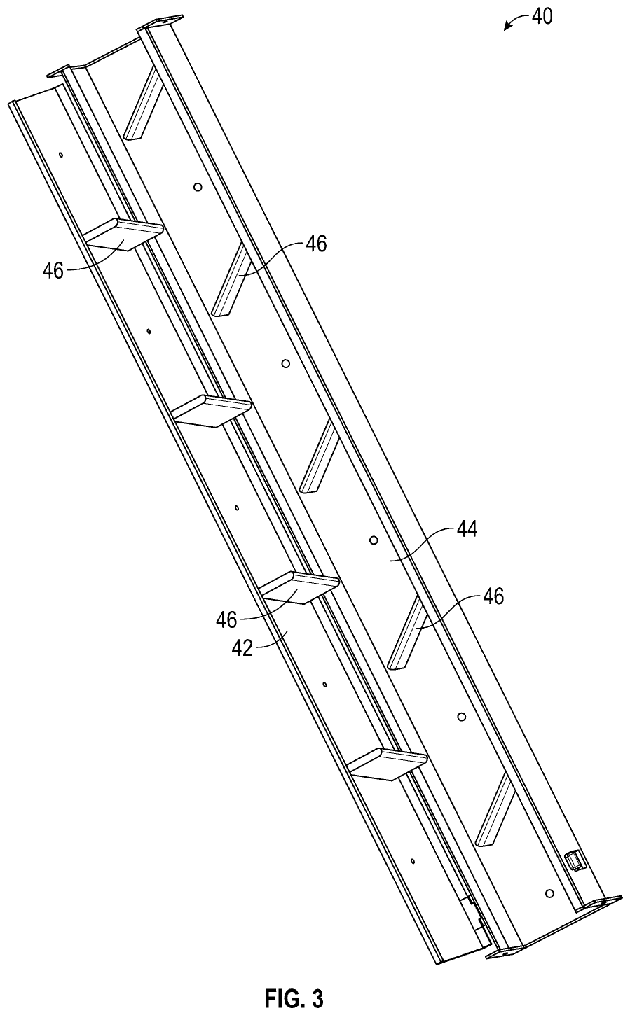

Turning now to FIG. 2, in an embodiment, the condensing chamber 20 may be utilized in conjunction with a frame or housing 40 that causes the condensing chamber 20 to take on the tortuous and/or serpentine-like configuration. For example, in an embodiment, the housing 40 may include a generally rigid front panel or front frame member 42 and a generally rigid rear panel or rear frame member 44 hingedly connected to the front panel 42. In an embodiment, one or both of the front panel 42 and the rear panel 44 may have a plurality of impaction members or features in the form of raised ridges or bars 46 protruding from the inner surface(s) thereof. In an embodiment, the ridges 46 are oriented generally horizontally. With reference to FIG. 3, in another embodiment, the ridges or bars 46 are oriented at an angle relative to horizontal. In either case, the ridges on the front panel 42 are staggered in the vertical direction with respect to the ridges of the rear panel 44.

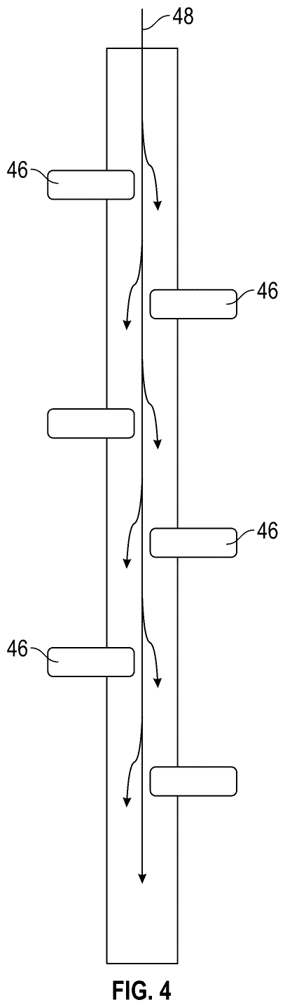

In use, the housing 40 is positioned adjacent to the bioreactor vessel 12 and the condensing chamber 20 is placed within the housing 40. The front panel/door 42 is then closed against the rear panel 44 to sandwich the condensing chamber 20 within the housing 40. In this position, as show in FIG. 4, the ridges 46 push the condensing chamber 20 away from a centerline through the housing 40, causing the condensing chamber 20 to take on a serpentine-like shape, creating the tortuous pathway within the condensing chamber 20 through which the exhaust gas 48 flows.

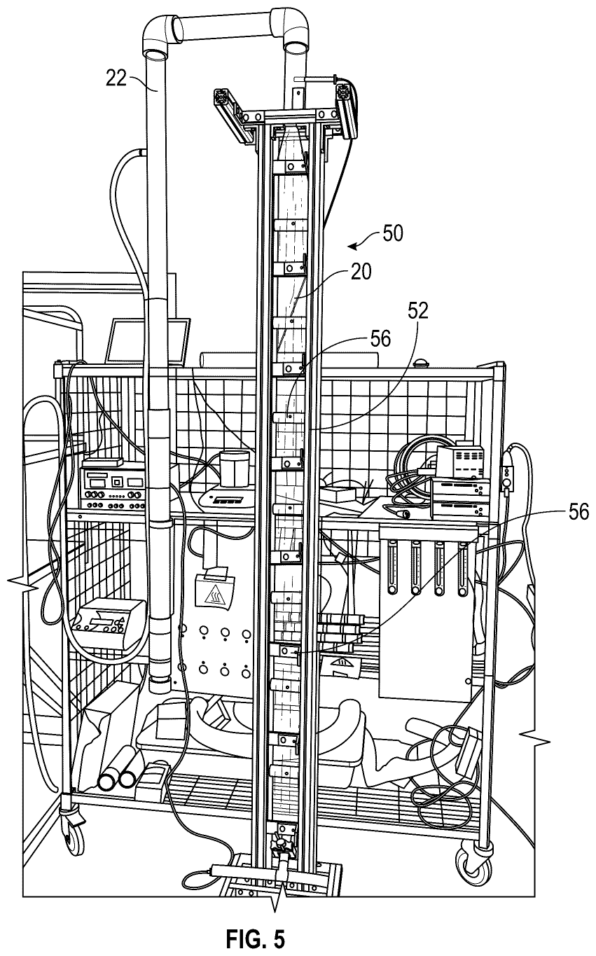

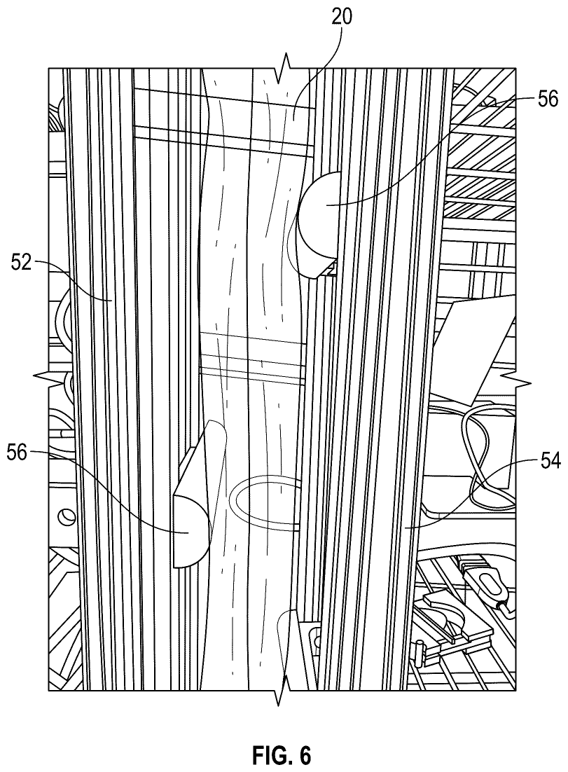

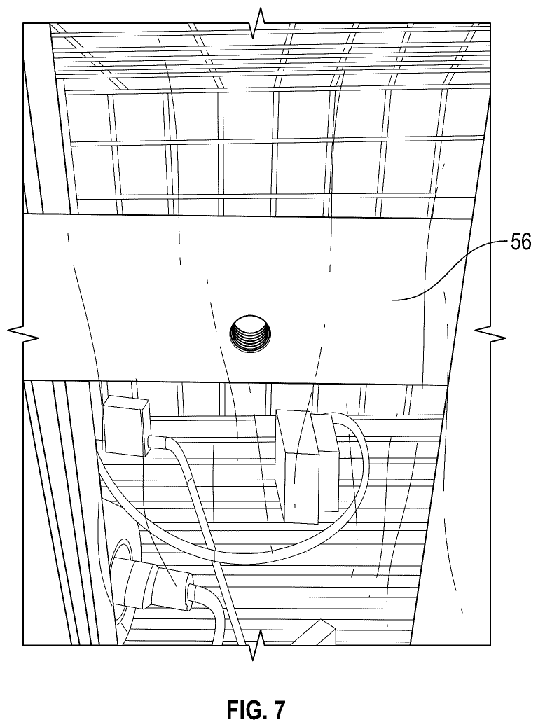

FIGS. 5-7 show another embodiment of a frame or housing 50 that can be utilized in conjunction with the condensing chamber 20 to form the tortuous pathway within the condensing chamber 20. As shown therein, the housing includes a front panel 52, a rear panel 54, and a plurality of impaction features in the form of horizontal bars or ridges 56. As best shown in FIG. 6, the ridges 56 deform or bias the thin-walled condensing container 20 so that the pathway therethrough takes on a serpentine or tortuous configuration.

As disclosed above, the impaction members impart the tortuous shape of the flexible container 20 by contacting and biasing the exterior of the container 20, and thus create the tortuous pathway within the thin-walled flexible condensing container 20, which facilitates droplet formation and particulate matter impaction onto the thin-walled container as the exhaust gas passes therethrough.

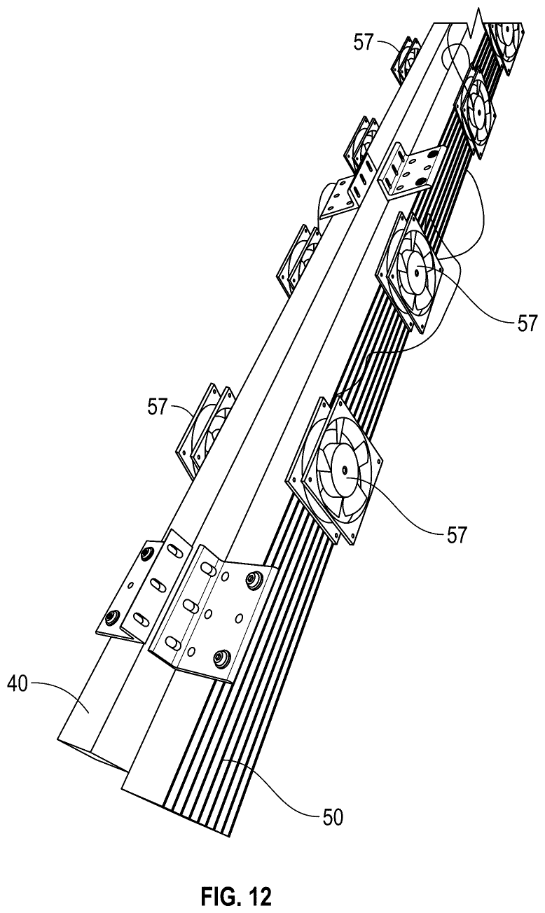

In any of the above-disclosed embodiments, the impaction members 46, 56 may be spring biased (or biased be similar means) towards the centerline of the respective housings 40, 50 to apply constant pressure to the condensing container 20 at different exhaust gas flow rates (and different condensing container inflation rates). Moreover, in any of the above-described embodiments, the system may further include one or more fans positioned above, below, or on the sides of the housing or frame members 40, 50 so as to flow ambient air across the exterior of the condensing container 20 (i.e., between the front and rear members and the condensing container 20 sandwiched therebetween) to cool the walls of the container 20 and thus facilitate condensation within the container 20. In this respect, external air cooling may be used to create a greater temperature gradient between the bag walls and the moist exhaust gas flowing through the container 20, to facilitate condensation and moisture removal, but still omit more costly and expensive active cooling elements (e.g., TCUs). For example, FIG. 12 shows a plurality of fans 57 mounted at various vertical locations along the housings 40, 50 for cooling the container 20 placed therein via convection airflow.

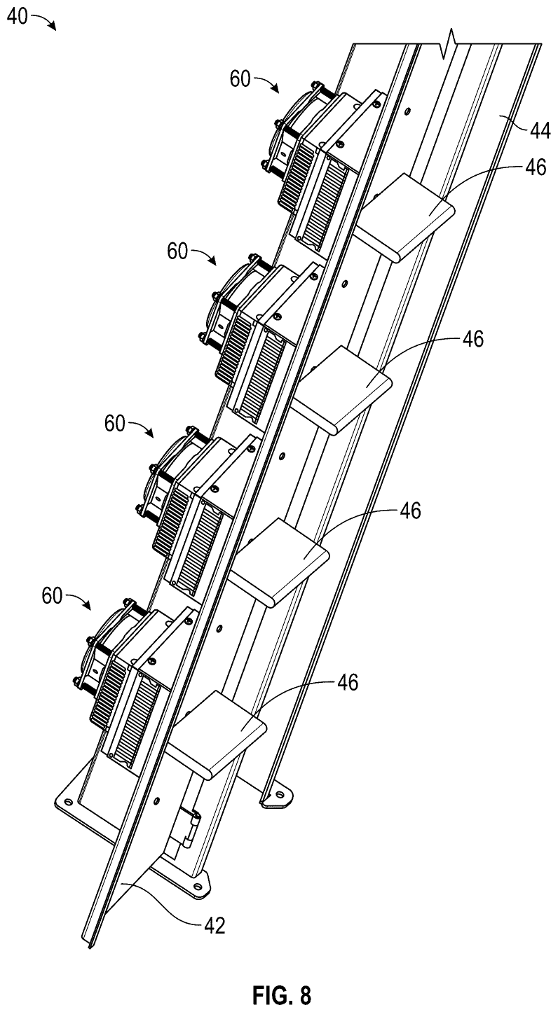

In an embodiment, the impaction members 46, 56 may be actively cooled so as to further facilitate condensation (by creating a greater temperature different between the condensing chamber 20 and the walls thereof, and the exhaust gas flowing therethrough). For example, in an embodiment, the impaction members 46, 56 may be Peltier-cooled. FIG. 8 illustrates such an embodiment, where the front and or rear panel 42, 44 of the housing 40 includes a plurality of thermoelectric cooling devices 60 such as, for example, Peltier cooling devices. While Peltier cooling devices 60 are illustrated, it is not intended that the invention be so limited in this regard. In particular, it is envisioned that a variety of active cooling technologies and devices may be utilized to cool the impaction members 46, 56 which contact and actively cool the flexible condensing container 20. The impaction members 46, 56 impart cooling to the condensing chamber 20 via direct contact with the exterior surface thereof.

In any of the above-described embodiments, the tortuous pathway within the bag created by the bag construction itself and/or the impaction members may extend either side-to-side, or front-to-back, or both side-to-side and front-to-back.

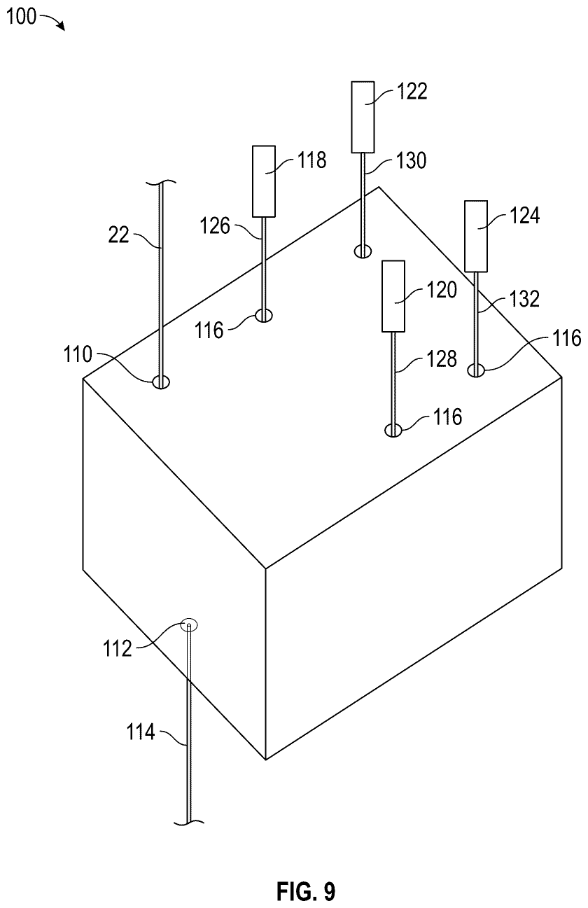

Referring now to FIG. 9, a condensing chamber 100 according to another embodiment of the invention is illustrated. The condensing chamber 100 is intended for use with the bioprocessing system of FIG. 1, and can be utilized in place of/instead of condensing chamber 20. As illustrated in FIG. 9, the condensing chamber 100 takes the form of a three-dimensional container or manifold. In an embodiment, the condensing chamber 100 may be a flexible bag configured to inflate when supplied with an exhaust gas, however, in other embodiments, the condensing chamber 100 may be rigid or semi-rigid. As shown in FIG. 9, the condensing chamber 100 has an inlet port 110 in a top surface thereof for fluid connection with the exhaust gas line 22 of bioprocessing system 10, and an outlet port or drain port 112 in a bottom surface thereof for fluid connection with a flow line or conduit 114 in fluid communication with the condensate accumulator 24. As also shown in FIG. 9, the condensing chamber 100 includes one or more ports 116 in the stop surface thereof to which exhaust filters 118, 120, 122, 124 are fluidly connected via dedicated flow lines 126, 128, 130, 132. While FIG. 9 illustrates four outlet ports 116 and four dedicated filters 118, 120, 122, 124, more or fewer than four ports, flow lines and associated filters may be utilized. In an embodiment, the outlet ports 116 are arranged in a square array. In an embodiment, each of the flow lines 126, 128, 130, 132 may include a sterile air filter and/or a valve for selectively allowing or preventing fluid communication with each filter with the interior volume of the condensing chamber 100. Similar to the condensing chamber 20, condensing chamber 100 is intended to be passively cooled simply by locating the condensing chamber 100 within the same ambient environment within which the bioprocessing system 10 is located. In an embodiment, the exhaust line 22 extends for at least about 3000 millimeters prior to inlet port 110, which ensures a sufficient residence time within the exhaust line 22 for initial cooling to facilitate condensation and moisture removal. Other configurations are possible depending on application and bioprocessing system operating parameters.

In operation, exhaust gas from headspace 18 of the bioprocessing bag 14 is routed to the condensing chamber 100 through exhaust line 22, where it enters the interior volume of the condensing chamber 100 through the top panel. As the exhaust gas and any entrained foam or particulate matter enter the condensing chamber 100, the foam and particulate matter tend to drop to the bottom of the condensing chamber 100 under the force of gravity and impaction. Moreover, the expansive volume of the condensing chamber 100 compared to the narrow exhaust line 22, the cooler environment within the condensing chamber 100, and the location of the outlet ports 116 on the same top panel as the inlet port 112 provide a reliable environment to facilitate condensation of moisture out of the moist exhaust gas stream. In particular, the expansive interior volume as compared to the narrow exhaust gas line 22 and the location of the inlet port 110 relative to the outlet ports 116 in the same top surface of the condensing chamber 100 increase the residence time of the moist exhaust gas within the condensing chamber 100, which facilitates condensation. As the moisture is removed from the exhaust gas, the accumulated moisture, foam and any particulate matter can be drained from the condensing chamber 100 through drain port 112 and line 114, where it can be temporarily collected in the condensate accumulator 24 and then either recycled to the bioreactor bag 14 or discarded. The dry exhaust gas can be exhausted through the respective filters 118, 120, 122 and/or 124.

In an embodiment, all of the filters 118, 120, 122, 124 can be used simultaneously, by opening all of the valves along flow lines 126, 128, 130, 132, although any combination of filters may be utilized by selectively opening or closing the associated valves. For example, in an embodiment, filters 118 and 120 may be used (by opening the associated valves), while filters 122 and 124 are inactive (by closing the associated valves), and vice versa, which has been discovered to provide for a flow balance between the utilized filters. In one embodiment, the ports corresponding to the filters 118 and 122 are approximately 200 millimeters apart, while the ports corresponding to filters 122 and 124 are approximately 200 millimeters apart. In addition, in an embodiment, the inlet port 110 is located approximately 100 millimeters from the port 116 associated with filter 118. Other configurations and arrangements are possible without departing from the broader aspects of the invention.

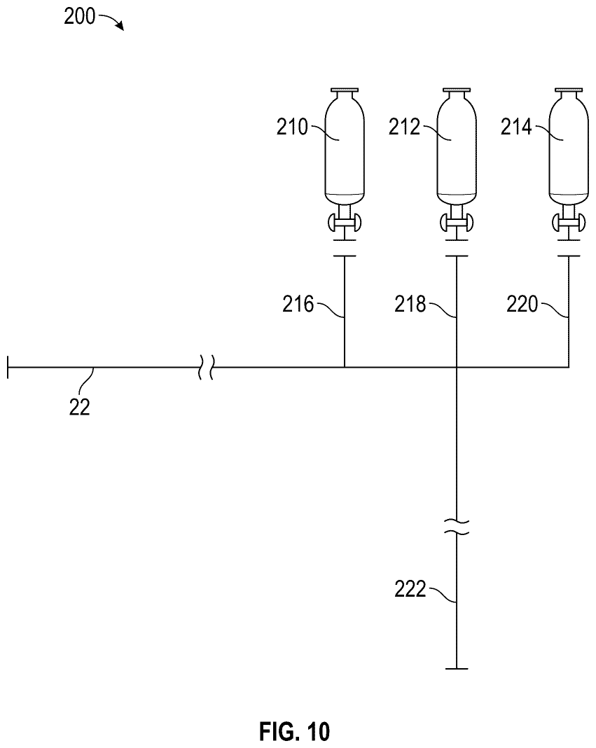

Referring to FIG. 10, a system 200 for condensation management for use with the bioprocessing system 10 of FIG. 1 is illustrated. As shown therein, the system 200 includes a plurality of exhaust filters 210, 212, 214 fluidly connected to the exhaust line 22 by branch conduits 216, 218, 220. While three exhaust gas filters are illustrated in FIG. 10, the invention is not intended to be so limited in this regard, and more than three filters or fewer than three filters (including as few as a single filter) may be utilized without departing from the broader aspects of the invention. In an embodiment, each of the conduits 216, 218, 220 may include a control valve (not shown) for selectively allowing or preventing flow through the conduits 216, 218, 220 to the associated filters 210, 212, 214. In an embodiment, the branch conduits 216, 218, 220 are oriented at an angle of about 90 degrees relative to the exhaust line 22 which inhibits any foam or particulate matter, and/or moist exhaust gas, from passing directly to the filters 210, 212, 214, and helps ensure an optimal residence time of the exhaust gas before reaching the filters. In an embodiment, liquid condensate entrainment is inhibited by sizing the branch conduit internal diameters to be large enough to reduce the upward gas velocity at maximum flow rates. For example, in an embodiment, the branch conduits 216, 218, 220 have an internal diameter of about 1 inch and the system 200 has a maximum exhaust gas flow rate of about 150 standard liter per minute. In such embodiment, the branch conduits 216, 218, 220 may have an internal diameter that is equal to or greater than the exhaust line 22.

In an embodiment, the junctions of the branch conduits 216, 218, 220 with the exhaust line 22 are spaced at intervals of about 300 millimeters, although other configurations are envisioned. In an embodiment the intervals are substantially equal, although the utilization of different intervals is also contemplated. In an embodiment, the exhaust line 22 extends for at least about 2000 millimeters prior to branch conduit 216, which ensures a sufficient residence time within the exhaust line 22 to facilitate condensation and moisture removal. Other configurations are possible depending on application and bioprocessing system operating parameters. As further shown in FIG. 10, the system 200 also includes a drain line 222 fluidly connected to the exhaust line 22 at a point intermediate the branch lines 216, 220. The drain line 222 extends downwardly and is in fluid communication with the condensate accumulator 24.

Similar to the embodiments disclosed above, moisture is removed from the exhaust gas stream via passive cooling, using the residence time within the exhaust line 22 to cool the exhaust gas. The even gas flow through the exhaust line 22 lowers the gas velocity, preventing liquid condensate entrainment in the venting line (which is possible when gas velocity exceeds draining speed). As the moisture is removed from the exhaust gas, the accumulated moisture, foam and any particulate matter can be drained from the condensing chamber 100 through drain line 222, where it can be temporarily collected in the condensate accumulator 24 and then either recycled to the bioreactor bag 14 or discarded. The dry exhaust gas is then exhausted through one or more of the filters 210, 212, 214.

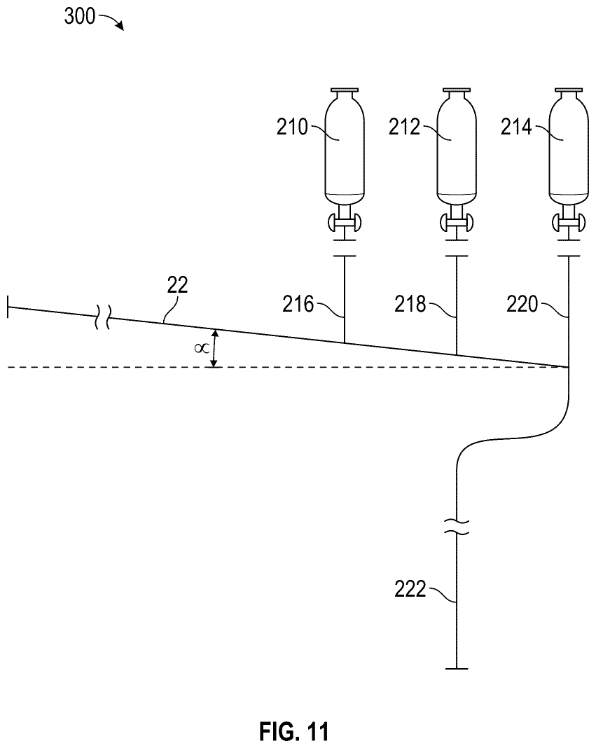

Referring finally to FIG. 11, a system 300 for condensation management for use with the bioprocessing system 10 of FIG. 1 is illustrated. As shown therein, the system 300 is substantially similar to system 200, where like reference numbers designate like parts. As shown therein, however, exhaust line 22 extends at a downward angle to facilitate the draining of condensate via gravity, and the drain line 222 is fluidly connected to the exhaust line 22 at the terminal end of the exhaust line 22. In an embodiment, the exhaust line may extend at a downward angle, α, between about 0 degrees and about 20 degrees. In an embodiment, the exhaust line extends at a downward angle of about 8 degrees.

Embodiments of the invention thus provided systems and methods for removing foam, moisture and particulate matter from an exhaust gas stream, thus providing a dry exhaust gas to the filters. This minimizes the potential for filter fouling, increasing the life of the filters and thus decreasing the operational costs of the bioprocessing system 10, as a whole. While certain embodiments of the invention contemplate utilizing active cooling to decrease the temperature of the condensing chamber or conduits within which condensation takes place, the invention functions well utilizing only passive cooling, namely, by utilizing the relatively lower temperature of the ambient environment to achieve the necessary temperature delta to effect condensation. The embodiments of the invention disclosed herein are thus much simpler and less costly to operate than existing condensation management systems and methods.

In any of the embodiments disclosed above, the filters that receive the dry gas after removal of moisture, particulate matter and/or foam, may be mounted/located at a height that is easily accessible from ground level for easy and ergonomic setup, servicing, replacement or changeover. For example, in an embodiment, the filters may be located between about 0 feet and about 8 feet above ground level. In other embodiments, the filters may be located between about 3 feet and about 7 feet above ground level. In yet other embodiments, the filters may be located between about 4 feet and about 6 feet above ground level. In an embodiment, the filters are located at about 5 feet above ground level. This filter location can be utilized in any bioprocessing system of the type disclosed herein regardless of capacity (e.g., 50 L, 500 L, 2000 L or more). This arrangement is facilitated by the use of the condensing chambers which extend down to (or can be located) near ground level. This, in contrast to existing systems which typically locate the filters above the bioprocessing vessel or support structure, removes the need for a ladder for filter setup, servicing and replacement, thereby greatly simplifying and improving usability of the overall bioprocessing system 10. Moreover, by having the filters accessible at floor level, the overall height of the bioprocessing system 10 is reduced, thereby also advantageously reducing the maximum room height required for operation.

Moreover, in any of the embodiments disclosed herein, one or more (or all) of the exhaust filters may be actively heated to reduce the relative humidity of the gas through the filter, providing a larger buffer against surface condensation on the filter membrane.

While embodiments of the invention have been described herein in connection with removing moisture from an exhaust gas stream of a bioprocessing system, it is not intended that the invention be so limited in this regard. In particular, it is contemplated that the techniques and methods disclosed herein may be utilized to remove moisture and particulate matter from any gas stream.

According to an embodiment of the invention, a method for removing moisture from a gas stream from a bioprocessing vessel includes the steps of passing an exhaust gas through a condensing chamber, the condensing chamber being passively cooled by an ambient environment within which the condensing chamber is located, a temperature of the condensing chamber causing moisture within the exhaust gas to condense within the condensing chamber to produce condensate, and passing the exhaust gas through at least one filter device. In an embodiment the method may further include the steps of removing the exhaust gas from a headspace within a bioprocessing vessel before passing the exhaust gas through the condensing chamber, and routing the collected condensate from the condensing chamber into the bioprocessing vessel. In an embodiment, the condensing chamber is a flexible bag. In an embodiment, the flexible bag has a tortuous pathway through which the exhaust gas travels. In an embodiment, the flexible bag is a two-dimensional flexible bag. In an embodiment, the method further includes the step of initiating a flow of ambient air along an outer surface of the flexible bag. In an embodiment, the method may include positioning the flexible bag within a frame, the frame having a front frame member and a rear frame member. At least one of the front frame member and the rear frame member includes a plurality of impaction members configured to contact the flexible bag and deform a shape of the flexible bag to form the tortuous pathway within the flexible bag. In an embodiment, both the front frame member and the rear frame member include a plurality of impaction members, wherein the impaction members of the front frame member are offset in a vertical direction from the impaction members of the rear frame member. In an embodiment, the method further includes the step of actively cooling the plurality of impaction members. In an embodiment, the impaction members are Peltier-cooled. In an embodiment, the plurality of impaction members are spring biased towards the flexible bag. In an embodiment, the plurality of impaction members are oriented at an angle. In an embodiment, the flexible bag includes a series of welds that define the tortuous pathway within the flexible bag. In an embodiment, the condensing chamber is a three-dimensional chamber having an inlet in a top side thereof for receiving the exhaust gas from the headspace of the bioprocessing vessel, and at least one outlet in a top side, the outlet being fluidly connected to the at least one filter device. In an embodiment, the at least one outlet is a plurality of outlets in the top side of the condensing chamber.

According to another embodiment of the invention, as system for condensation management in a bioprocessing system includes a condensing chamber having an inlet configured for fluid connection with a headspace of a bioprocessing vessel, and an outlet configured for fluid connection with an exhaust filter, wherein the condensing chamber is configured to be passively cooled by an ambient environment within which the condensing chamber and the bioprocessing vessel are located. In an embodiment, the condensing chamber is a flexible bag having a tortuous pathway between the inlet and the outlet. In an embodiment, the system further includes a frame having a front frame member and a rear frame member, and a plurality of impaction members on at least one of the front frame member and the rear frame member, wherein the frame is configured to receive the flexible bag intermediate the front frame member and the rear frame member, and wherein the plurality of impaction members are configured to contact the flexible bag and deform a shape of the flexible bag to form the tortuous pathway within the flexible bag. In an embodiment, both the front frame member and the rear frame member include a plurality of impaction members, wherein the impaction members of the front frame member are offset in a vertical direction from the impaction members of the rear frame member. In an embodiment, the system additionally includes a thermoelectric cooling device configured to cool the plurality of impact members below a temperature of the ambient environment. In an embodiment, the impaction members are spring biased towards the flexible bag. In an embodiment, the system additionally includes a fan configured to generate a flow of ambient air along an outer surface of the flexible bag. In an embodiment, the flexible bag includes a series of welds that define the tortuous pathway within the flexible bag. In an embodiment, the condensing chamber is a three-dimensional chamber, and the inlet is located in a top side of the condensing chamber. In an embodiment, the outlet is a plurality of outlets located in the top side of the condensing chamber. In an embodiment, the condensing chamber includes a drain in a bottom side of the condensing chamber. In an embodiment, the condensing chamber is a length of tubing, the outlet is a plurality of outlets in the length of tubing, and the system further includes a plurality of branch conduits extending from the length of tubing and fluidly connected with the length of tubing the plurality of outlets. In an embodiment, the system may further include a filter element fluidly connected with each of the branch conduits. In an embodiment, the system may also include a drain line fluidly connected to the length of tubing. In an embodiment, the system also includes the exhaust filter, wherein the exhaust filter is positioned between about 4 feet and about 7 feet above ground level.

According to yet another embodiment of the invention, a method for removing moisture from a gas stream during a bioprocessing operation is provided. The method includes the steps of removing an exhaust gas from a headspace within a bioprocessing vessel, and passing the exhaust gas through a condensing chamber, the condensing chamber being passively cooled by an ambient environment within which the condensing chamber is located, a temperature of the condensing chamber causing moisture within the exhaust gas to condense within the condensing chamber to produce condensate. The method further includes the steps of passing the exhaust gas through at least one filter device, and routing the collected condensate from the condensing chamber into the bioprocessing vessel.

As used herein, an element or step recited in the singular and proceeded with the word “a” or “an” should be understood as not excluding plural of said elements or steps, unless such exclusion is explicitly stated. Furthermore, references to “one embodiment” of the invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A method for removing moisture from a gas stream from a bioprocessing vessel, comprising the steps of:
passing an exhaust gas through a condensing chamber, the condensing chamber being passively cooled by an ambient environment within which the condensing chamber is located, a temperature of the condensing chamber causing moisture within the exhaust gas to condense within the condensing chamber to produce condensate; and
passing the exhaust gas through at least one filter device.

2. The method according to claim 1, further comprising the steps of:
removing the exhaust gas from a headspace within a bioprocessing vessel before passing the exhaust gas through the condensing chamber; and
routing the collected condensate from the condensing chamber into the bioprocessing vessel.

3. The method according to claim 1, wherein:
the condensing chamber is a flexible bag.

4. The method according to claim 3, wherein:
the flexible bag has a tortuous pathway through which the exhaust gas travels.

5. The method according to claim 3, wherein:
the flexible bag is a two-dimensional flexible bag.

6. The method according to claim 3, further comprising the step of:
initiating a flow of ambient air along an outer surface of the flexible bag.

7. The method according to claim 4, further comprising the step of:
positioning the flexible bag within a frame, the frame having a front frame member and a rear frame member;
wherein at least one of the front frame member and the rear frame member includes a plurality of impaction members configured to contact the flexible bag and deform a shape of the flexible bag to form the tortuous pathway within the flexible bag.

8. The method according to claim 7, wherein:
both the front frame member and the rear frame member include a plurality of impaction members;
wherein the impaction members of the front frame member are offset in a vertical direction from the impaction members of the rear frame member.

9. The method according to claim 7, further comprising the step of:
actively cooling the plurality of impaction members.

10. The method according to claim 9, wherein:
the impaction members are Peltier-cooled.

11. The method according to claim 7, wherein:
the plurality of impaction members are spring biased towards the flexible bag.

12. The method according to claim 7, wherein:
the plurality of impaction members are oriented at an angle.

13. The method according to claim 4, wherein:
the flexible bag includes a series of welds that define the tortuous pathway within the flexible bag.

14. The method according to claim 2, wherein:
the condensing chamber is a three-dimensional chamber having an inlet in a top side thereof for receiving the exhaust gas from the headspace of the bioprocessing vessel, and at least one outlet in a top side, the outlet being fluidly connected to the at least one filter device.

15. The method according to claim 14, wherein:
the at least one outlet is a plurality of outlets in the top side of the condensing chamber.

16. A system for condensation management in a bioprocessing system, comprising:
a condensing chamber having an inlet configured for fluid connection with a headspace of a bioprocessing vessel, and an outlet configured for fluid connection with an exhaust filter; and
wherein the condensing chamber is configured to be passively cooled by an ambient environment within which the condensing chamber and the bioprocessing vessel are located.

17. The system for condensation management of claim 16, wherein:
the condensing chamber is a flexible bag having a tortuous pathway between the inlet and the outlet.

18. The system for condensation management of claim 17, further comprising:
a frame having a front frame member and a rear frame member, and a plurality of impaction members on at least one of the front frame member and the rear frame member;
wherein the frame is configured to receive the flexible bag intermediate the front frame member and the rear frame member; and
wherein the plurality of impaction members are configured to contact the flexible bag and deform a shape of the flexible bag to form the tortuous pathway within the flexible bag.

19. The system for condensation management of claim 18, wherein:
both the front frame member and the rear frame member include a plurality of impaction members;
wherein the impaction members of the front frame member are offset in a vertical direction from the impaction members of the rear frame member.

20. The system for condensation management of claim 18, further comprising:
a thermoelectric cooling device configured to cool the plurality of impact members below a temperature of the ambient environment.

21. The system condensation management of claim 18, wherein:
the impaction members are spring biased towards the flexible bag.

22. The system for condensation management of claim 17, further comprising:
a fan configured to generate a flow of ambient air along an outer surface of the flexible bag.

23. The system for condensation management of claim 17, wherein:
the flexible bag includes a series of welds that define the tortuous pathway within the flexible bag.

24. The system for condensation management of claim 16, wherein:
the condensing chamber is a three-dimensional chamber;
wherein the inlet is located in a top side of the condensing chamber.

25. The system for condensation management of claim 23, wherein:
the outlet is a plurality of outlets located in the top side of the condensing chamber.

26. The system for condensation management of claim 23, wherein:
the condensing chamber includes a drain in a bottom side of the condensing chamber.

27. The system for condensation management of claim 16, wherein:
the condensing chamber is a length of tubing; and
wherein the outlet is a plurality of outlets in the length of tubing; and
wherein the system further includes a plurality of branch conduits extending from the length of tubing and fluidly connected with the length of tubing the plurality of outlets.

28. The system for condensation management of claim 27, further comprising:
a filter element fluidly connected with each of the branch conduits.

29. The system for condensation management of claim 28, further comprising:
a drain line fluidly connected to the length of tubing.

30. The system for condensation management of claim 28, further comprising:
the exhaust filter;
wherein the exhaust filter is positioned between about 4 feet and about 7 feet above ground level.

31. A method for removing moisture from a gas stream during a bioprocessing operation, comprising the steps of:
removing an exhaust gas from a headspace within a bioprocessing vessel;
passing the exhaust gas through a condensing chamber, the condensing chamber being passively cooled by an ambient environment within which the condensing chamber is located, a temperature of the condensing chamber causing moisture within the exhaust gas to condense within the condensing chamber to produce condensate;
passing the exhaust gas through at least one filter device; and
routing the collected condensate from the condensing chamber into the bioprocessing vessel.
